(19) United States
(12) Patent Application Publication
(10) Pub. No.: US 2026/0115151 A1
(43) Pub. Date: Apr. 30, 2026

(54) VITAMIN AND MINERAL SHOT

(71) Applicant: Corinne Barranco, Palm Beach Gardens, FL (US)

(72) Inventor: Corinne Barranco, Palm Beach Gardens, FL (US)

(21) Appl. No.: 19/371,400

(22) Filed: Oct. 28, 2025

Related U.S. Application Data
(60) Provisional application No. 63/714,645, filed on Oct. 31, 2024.

(51) Int. Cl. A61K 31/07; A23L 2/56; A23L 2/60; A61K 9/00; A61K 31/122; A61K 31/14; A61K 31/197; A61K 31/355; A61K 31/375; A61K 31/4188; A61K 31/4415; A61K 31/455; A61K 31/51; A61K 31/519; A61K 31/522; A61K 31/525; A61K 31/593; A61K 31/714; A61K 33/04; A61K 33/06; A61K 33/18; A61K 33/24; A61K 33/26; A61K 33/30; A61K 33/32; A61K 33/34; A61K 33/42; A61K 36/82; A61K 47/12

(52) U.S. Cl. CPC A61K 31/07; A23L 2/56; A23L 2/60; A61K 9/0095; A61K 31/122; A61K 31/14; A61K 31/197; A61K 31/355; A61K 31/375; A61K 31/4188; A61K 31/4415; A61K 31/455; A61K 31/51; A61K 31/519; A61K 31/522; A61K 31/525; A61K 31/593; A61K 31/714; A61K 33/04; A61K 33/06; A61K 33/18; A61K 33/24; A61K 33/26; A61K 33/30; A61K 33/32; A61K 33/34; A61K 33/42; A61K 36/82; A61K 47/12

(57) ABSTRACT

A vitamin shot having essential vitamins, minerals and a plant extract.

### FIELD OF THE INVENTION

This invention relates to a daily essentials supplement. More specifically, a liquid shot including vitamins, minerals, and energy.

### BACKGROUND OF THE INVENTION

Conventional supplements and vitamins have deficiencies causing people to not feel their absolute best. In a busy world/lifestyle, a person does not always have time to take care of their body. Multiple supplements must be taken multiple times a day.

U.S. Published patent application No. 20080193604 (Melton) discloses a supplement drink in the form of a shot and how to make the supplement drink. The entire disclosure of this application is incorporated herein by reference. The present vitamin and mineral shot can be produced in a similar manner.

As a nurse advising patients to take multiple supplements a day, while rarely providing 100% of what they truly need, the demand for a single product that wholly offers everything adults need in a day is needed. Thus, there is a need for a quick vitamin shot having essential vitamins and minerals.

### SUMMARY OF THE INVENTION

An objective of the invention is to provide a vitamin shot that provides essential vitamins, minerals, and energy to a human in a fast and convenient manner.

Another objective is to include at least one natural plant extract.

This objective and other objectives can be obtained by a vitamin shot comprising a carrier, essential vitamins, minerals, plant extracts, and caffeine.

Very few people forget their energy source in the morning, such as coffee or tea. The present invention now provides necessary vitamins and minerals. A commercial embodiment is envisioned as Your Essential Shot, which brings good health and positive energy by offering the vitamins and minerals needed each day. One single shot a day to keep the doctor away. Deliciously flavored with organic extracts and organic monk fruit as a natural, sugar free sweetener. With the addition of energy/caffeine this is the only supplement adults need in the morning to reveal their best and healthiest self.

Objectives of the invention can be obtained by a vitamin shot comprising:
an effective amount of vitamins A, B1, B2, B3, B5, B6, B7, B9, B12, C, D3, E, and K2;
an effective amount of minerals Zinc, Iodine, Copper, Calcium, Choline, Iron, Magnesium, Manganese, Selenium, and Chromium and Phosphorous;
an extract; and
a carrier in amount to provide the shot with a volume of about 1-3 oz, preferably 2.5 oz.

### DETAILED DESCRIPTION OF THE INVENTION

The invention will be explained with reference to specific embodiments, without being limited thereto.

“A” or “an” means “at least one” or “one or more” unless otherwise indicated.

When referring to a group, “at least one . . . and . . . ” in the specification and claims is synonymous with “and/or”. For example, “at least one of A, B and C” means A alone, B alone, C alone, or any combination of A, B or C.

“Comprise”, “have”, “include” and “contain” (and their variants) are open-ended linking verbs and allow the addition of other elements when used in a claim.

“Consisting of” is closed, and excludes all additional elements.

“Consisting essentially of” excludes additional material elements, but allows the inclusions of non-material elements that do not substantially change the nature of the invention.

The term “shot” refers to an amount of liquid a human can consume at one serving, about 1-3 oz, preferably 2.5 oz. The shot is preferably sized for easy swallowing.

The vitamin shot preferably contains the following ingredients dissolved or dispersed in a carrier in one shot:

TABLE 1

| YES Formulation | Acceptable Ranges |
|---|---|
| Vitamin A (Beta-Carotene) | 700 mcg RAE-900 mcg RAE |
| 900 mcg RAE = 100% DV | |
| B1: Thiamin (Thiamine Hydrochloride) | 1.2 mg-3 mg |
| 1.2 mg = 100% | |
| B2: Riboflavin | 1.3 mg-3.25 mg |
| 1.3 mg = 100% | |
| B3: Niacin (Niacinamide) | 16 mg NE-40 mg NE |
| 16 mg NE = 100% | |
| B5: Pantothenic Acid | 5 mg-12.5 mg |
| (Calcium D-Pantothenate) | |
| 5 mg = 100% | |
| B6: Pyridoxine (Pyridoxine | 4.25 mg-8.5 mg |
| Hydrochloride) | |
| 4.25 mg = 250% | |
| B7: Biotin | 30 mcg-75 mcg |
| 30 mcg = 100% | |
| B9: Folate (Calcium L-5 | 400 mcg DFE-600 mcg DFE |
| Methyltetrahydrofolate) | |
| 400 mcg DFE = 100% | |
| B12: Methylcobalamin | 6 mcg-15 mcg |
| 6 mcg = 250% | |
| Vitamin C (Ascorbic Acid) | 180 mg-450 mg |
| 180 mg = 200% | |
| Vitamin D3 (Cholecalciferol) | 20 mcg [800 iu]-50 mcg |
| 25 mcg [1000 iu] = 125% | [2000 iu] |
| Vitamin E (D-alpha-tocopherol acetate) | 15 mg-22.5 mg |
| 15 mg [22.4 iu] = 100% | |
| Vitamin K2 (Menaquinone-7) | 90 mcg-180 mcg |
| 120 mcg = 100% | |
| Zinc ( Zinc Glycinate) | 8.25 mg-11 mg |
| 8.25 mg = 75% | |
| Iodine (Potassium Iodide) | 37.5 mcg-187.5 mcg |
| 37.5 mcg = 25% | |
| Copper (Copper Bisglycinate) | 450 mcg-900 mcg |
| 450 mcg = 50% | |
| Calcium (Calcium Lactate) | 75 mg-130 mg |
| 130 mg = 10% | |
| Iron (Iron Bisglycinate) | 4.5 mg-18 mg |
| 4.5 mg = 25% | |
| Magnesium (Magnesium Bisglycinate) | 40 mg-50.4 mg |
| 40 mg = 10% | |
| Manganese (Manganese Gluconate) | 0.5 mg-2.2 mg |
| 0.58 mg = 25% | |
| Selenium (Selenomethionine) | 27.5 mcg-65 mcg |
| 27.5 mcg = 50% | |
| Chromium (Chromium Picolinate) | 25 mcg-53 mcg |
| 26.25 mcg = 75% | |
| Choline (Choline L-Bitartrate) | 27.5 mg-82.5 mg |
| 55 mg = 10% | |
| Phosphorus (Dipotassium Phosphate) | 35 mg-105 mg |
| 40 mg = 4% | |
| Organic Caffeine from Green Tea | 100 mg-130 mg |
| 100 mg | |
| Organic Flavor Extracts * | Amount to provide desired |
| | taste and/or smell. |

Carriers are now well-known and any suitable carrier can be utilized. Purified water is a preferred carrier.

Any suitable additive can be included as desired, such as binders, emulsifiers, maskers, stabilizers, sweeteners, preservatives, or other.

Any suitable organic flavor extracts can be utilized. Preferably, the extract is at least one of lavender and pomegranate. The extract can be added in an amount to provide the desired flavor and/or smell result.

### EXAMPLES

### Example 1

An example of a preferred vitamin shot formulation contains the following ingredients in one shot:
Vitamins
A: 900 mcg RAE—100% “Beta-Carotene”
C: 180 mg—200% “Ascorbic Acid”
D3: 25 mcg [1000 iu]—125% “Cholecalciferol”
E: 15 mg [22.4 iu]—100% “D-alpha-tocopherol acetate”
K2: 120 mcg—100% “Menaquinone-7”
B1: Thiamin: 1.2 mg—100% “Thiamine Hydrochloride”
B2: Riboflavin: 1.3 mg—100%
B3: Niacin: 16 mg NE—100% “Niacinamide”
B5: Pantothenic Acid: 5 mg—100% “Calcium D-Pantothenate”
B6: Pyridoxine: 4.25 mg—250% “Pyridoxine Hydrochloride”
B7: Biotin: 30 mcg—100%
B9: Folate: 400 mcg DFE—100% “Calcium L-5 Methyltetrahydrofolate”
B12: 6 mcg—250% “Methylcobalamin”
Minerals
Calcium: 130 mg—10% RDA “Calcium Lactate”
Iron: 4.5 mg—25% RDA “Iron Bisglycinate”
Iodine: 37.5 mcg—25% RDA “Potassium Iodide”
Magnesium: 40 mg—10% RDA “Magnesium Bisglycinate”
Zinc: 8.25 mg—75% RDA “Zinc Glycinate”
Selenium: 27.5 mcg—50% RDA “Selenomethionine”
Copper: 450 mcg—50% RDA “Copper Bisglycinate”
Manganese: 0.58 mg—25% RDA “Manganese Gluconate”
Chromium: 26.25 mcg—75% RDA “Chromium Picolinate”
Phosphorus: 40 mg—4% RDA “Dipotassium Phosphate”
Choline: 55 mg˜10% RDA “Choline L-Bitartrate”
Other Ingredients
Carrier: The preferred carrier is purified filtered water
Flavor Substance: Preferably a mixture of organic extracts. (Organic flavors 800 mg—2.5 g per shot), (Monk Fruit 55 mg—85 mg per shot) (Citric
Acid 355-460 mg per shot)
Energy: Organic caffeine from green tea 100 mg, which is equivalent to the amount of caffeine in 1 cup of coffee.

The pomegranate shot was formed mixing the ingredients together as follows. Add minerals to mix tank containing approximately 50% of total warm (approx. 100-110 F) water. Mix until completely dissolved using medium shear agitation. Add monk fruit, green tea, and vitamins to mix tank. Continuously mix until completely incorporated using medium shear agitation. Add an additional 20% of ambient (approx. 75 F) water to the tank followed by flavor and mix well. Mix until completely homogeneous using medium shear agitation. Finally, add citric acid and bring up to volume with the remaining ambient (approx. 75 F) water and mix well. Take specs to make sure product meets finished product specifications. Hot fill into final packaging.

### Laboratory Scale Specifications—Finished Product

Product Density: 8.456 lbs/gallon
pH: 4.10+/−0.20
Brix: 4.10+/−0.20
Taste: Pomegranate Characteristics
Appearance: Cloudy Orange Liquid

### Example 2

Another example of a preferred vitamin shot includes the following ingredients:

TABLE 2
Supplement Facts Serving Size 1 Shot

Amount Per Serving
% Daily Value
Vitamin A (Beta-Carotene)
900 mcg RAE 100%
B1: Thiamin (Thiamine Hydrochloride)
1.2 mg 100%
B2: Riboflavin
1.3 mg 100%
B3: Niacin (Niacinamide)
16 mg NE 100%
B5: Pantothenic Acid (Calcium
D-Pantothenate)
5 mg 100%
B6: Pyridoxine (Pyridoxine
Hydrochloride)
4.25 mg 250%
B7: Biotin
30 mcg 100%
B9: Folate (Calcium L-5
Methyltetrahydrofolate)
400 mcg DFE 100%
B12: Methylcobalamin
6 mcg 250%
Vitamin C (Ascorbic Acid)
180 mg 200%
Vitamin D3 (Cholecalciferol)
25 mcg [1000 iu] 125%
Vitamin E (D-alpha-tocopherol acetate)
15 mg [22.4 iu] 100%
Vitamin K2 (Menaquinone-7)
120 mcg 100%
Zinc ( Zinc Glycinate)
8.25 mg 75%
Iodine (Potassium Iodide)
37.5 mcg 25%
Copper (Copper Bisglycinate)
450 mcg 50%
Calcium (Calcium Lactate)
130 mg 10%
Iron (Iron Bisglycinate)
4.5 mg 25%
Magnesium (Magnesium Bisglycinate)
40 mg 10%
Manganese (Manganese Gluconate)
0.58 mg 25%
Selenium
(Selenomethionine)
27.5 mcg 50%
Chromium (Chromium Picolinate)
26.25 mcg 75%
Choline (Choline L-Bitartrate)
55 mg 10%
Phosphorus (Dipotassium Phosphate)
40 mg 4%
Organic Caffeine from Green Tea
100 mg **
Organic Extracts **
The organic extracts can be present
from 817 mg-2.5 g per shot.
Percent Daily Values are based on
a 2,000 calorie diet
** Daily Value not established
Other Ingredients: Purified Water,
Organic Monk Fruit Extract, Citric Acid,
Organic Masker. (Monk Fruit 55-85 mg
per shot) (Citric Acid 355-460 mg per
shot)
Gender Neutral, Made in USA, Vegan,
Gluten Free, Soy Free, Keto, Paleo,
Non-GMO, Zero Sugar, Zero Additives,
Zero Fillers.

The lavender shot was formed mixing the ingredients together as follows. Add minerals to mix tank containing approximately 50% of total warm (approx. 100-110 F) water. Mix until completely dissolved using medium shear agitation. Add monk fruit and vitamins to mix tank. Continuously mix until completely incorporated using medium shear agitation. Add an additional 20% of ambient (approx. 75 F) water to the tank followed by flavor and mix well. Mix until completely homogeneous using medium shear agitation. Finally, add citric acid and bring up to volume with the remaining ambient (approx. 75 F) water and mix well. Take specs to make sure product meets finished product specifications. The mixture was hot filled into final shot sized packaging.

### Laboratory Scale Specifications—Finished Product

Product Density: 8.442 lbs/gallon
PH: 4.18+/−0.20
Brix: 3.70+/−0.20
Taste: Lavender Characteristics
Appearance: Cloudy Orange Liquid

### Example 3

A further example of a preferred formulation is:

TABLE 3
LABORATORY SCALE FORMULA

| Ingredients | Percent |
|---|---|
| Filtered Water | 94.1932584 |
| Beta Carotene 1% Powder | 0.2430000 |
| Thiamine HCl (Vitamin B1) Powder | 0.0016200 |
| Riboflavin (Vitamin B2) Powder | 0.0017600 |
| Niacin (Vitamin B3) Powder | 0.0216000 |
| Vitamin B5 Pantothenic Acid/Calcium | 0.0074000 |
| Pantothenate Powder | |
| Pyridoxine HCl (Vitamin B6) Powder | 0.0057400 |
| Biotin 10000 mcg Powder | 0.0040500 |
| L-5-Methyltetrahydrofolate Calcium | 0.0007000 |
| Vitamin B12 1% (Methylcyanocobalamin) | 0.0008110 |
| Powder | |
| Ascorbic Acid (Vitamin C) Powder | 0.2430000 |
| CELLg8 ® Vegan Vitamin D3 | 0.0135000 |
| Vitamin E 400 IU Powder | 0.0362000 |
| Vitamin K2 M7 (1%) Powder | 0.0162000 |
| Zinc Glycinate Powder | 0.0362500 |
| Copper Bisglycinate | 0.0020200 |
| Calcium Lactate | 1.2500000 |
| Iron Bisglycinate Powder 20% | 0.0305000 |
| Magnesium Bisglycinate | 0.2850000 |
| Manganese Gluconate | 0.0071000 |
| L-Selenomethionine 5000 MD | 0.0074200 |
| (Selenium 0.5%) | |
| Chromium Picolinate Powder | 0.0003006 |
| Choline L-Bitartrate Powder | 0.0743200 |
| Dipotassium Phosphate | 0.3381000 |
| PurTea ® Caffeine from Green Tea | 0.1501500 |
| Citric Acid Powder | 0.4600000 |
| MonkSweet V50 \| Organic Monk | 0.0850000 |
| Fruit Extract | |
| Organic Certifiable Flavor Blend | 0.2500000 |
| (Vitamin Modifer Type) | |
| Sweetness Enhancer FI N Ty Org | 0.3500000 |
| Pom Berry FI Nat Type Org Cbl | 0.8200000 |
| Pomegranate FI Nat Type Org | 0.5150000 |
| Pomegranate FI WONF Nat Organic | 0.5500000 |
| Weight percent | 100.000 |

The pomegranate shot was formed by adding the minerals to a mix tank containing approximately 50% of total warm (approx. 100-110 F) water. The mixture was stirred until the minerals were completely dissolved using medium shear agitation. Monk fruit and vitamins were then added to the mix tank and continuously mixed until completely incorporated using medium shear agitation. An additional 20% of ambient (approx. 75 F) water was added to the mix tank followed by flavor and the mixture was mixed until completely homogeneous using medium shear agitation. Citric acid and water was added to the mix tank, the volume was increased with the remaining ambient (approx. 75 F) water, and the mixture was mixed well. Specifications were measured to make sure the product met finished product specifications. The mixture was hot filled into final shot sized packaging.

### Laboratory Scale Specifications—Finished Product

Product Density: 8.456 lbs/gallon
pH: 4.1+/−0.20
Brix: 4.1+/−0.20
Taste: Pomegranate Characteristics
Appearance: Cloudy Orange Liquid

### Example 4

Another example of a preferred formulation is:

TABLE 4
Serving Size: LABORATORY SCALE FORMULA

| Ingredients | Percent |
|---|---|
| Filtered Water | 95.7075804 |
| Beta Carotene 1% Powder | 0.2430000 |
| Thiamine HCl (Vitamin B1) Powder | 0.0016200 |
| Riboflavin (Vitamin B2) Powder | 0.0017600 |
| Niacin (Vitamin B3) Powder | 0.0216000 |
| Vitamin B5 Pantothenic Acid/Calcium | 0.0074000 |
| Pantothenate Powder | |
| Pyridoxine HCl (Vitamin B6) Powder | 0.0057400 |
| Biotin 10000 mcg Powder | 0.0040500 |
| L-5-Methyltetrahydrofolate Calcium | 0.0007000 |
| Vitamin B12 1% (Cyanocobalamin) Powder | 0.0008110 |
| Ascorbic Acid (Vitamin C) Powder | 0.2430000 |
| CELLg8 ® Vegan Vitamin D3 | 0.0135000 |
| Vitamin E 400 IU Powder | 0.0362000 |
| Vitamin K1 (1%) Powder | 0.0162000 |
| Zinc Glycinate Powder | 0.0362500 |
| Copper Bisglycinate | 0.0019500 |
| Calcium Lactate | 1.2500000 |
| Iron Bisglycinate Powder 20% | 0.0305000 |
| Magnesium Bisglycinate | 0.2850000 |
| Manganese Gluconate | 0.0071000 |
| L-Selenomethionine 5000 MD | 0.0074200 |
| (Selenium 0.5%) | |
| Chromium Picolinate Powder | 0.0003006 |
| Choline L-Bitartrate Powder | 0.0743200 |
| Dipotassium Phosphate | 0.3381000 |
| Potassium Iodine 5% | 0.0008980 |
| Citric Acid Powder | 0.4800000 |
| Organic Lavender Flavor WONF | 0.1750000 |
| Organic Lavender Extract Isoz | 0.4800000 |
| Organic Certifiable Flavor Blend | 0.2000000 |
| (Vitamin Modifer Type) | |
| Sweetness Enhancer FI N Ty Org | 0.2500000 |
| MonkSweet V50 \| Organic Monk | 0.0800000 |
| Fruit Extract | |
| Weight percent | 100.000 |

The lavender shot was formed by adding the minerals to a mix tank containing approximately 50% of total warm (approx. 100-110 F) water. The mixture was stirred until the minerals were completely dissolved using medium shear agitation. Monk fruit and vitamins were then added to the mix tank and continuously mixed until completely incorporated using medium shear agitation. An additional 20% of ambient (approx. 75 F) water was added to the mix tank followed by flavor and the mixture was mixed until completely homogeneous using medium shear agitation. Citric acid and water was added to the mix tank, the volume was increased with the remaining ambient (approx. 75 F) water, and the mixture was mixed well. Specifications were measured to make sure the product met finished product specifications. The mixture was hot filled into final shot sized packaging.

### Laboratory Scale Specifications—Finished Product

Product Density: 8.442 lbs/gallon
pH: 4.18+/−0.20
Brix: 3.70+/−0.20
Taste: Lavender Characteristics
Appearance: Cloudy Orange Liquid

While the invention has been described with reference to particular embodiments, it will be understood by those skilled in the art that various changes may be made and equivalents may be substituted for elements thereof without departing from the scope of the invention. In addition, many modifications may be made to adapt a particular situation or material to the teachings of the invention without departing from the scope of the invention.

Therefore, it is intended that the invention not be limited to the particular embodiments disclosed as the best mode contemplated for carrying out this invention, but that the invention will include all embodiments falling within the scope and spirit of the appended claims.

1. A vitamin shot comprising:
an effective amount of vitamins A, B1, B2, B3, B5, B6, B7, B9, B12, C, D3, E, and K2;
an effective amount of minerals Zinc, Iodine, Copper, Calcium, Choline, Iron, Magnesium, Manganese, Selenium, Chromium, and Phosphorous;
a plant extract; and
a carrier in amount to provide the shot with a volume of about 1-3 oz.

2. The vitamin shot according to claim 1, wherein the carrier is in an amount to provide the shot with a volume of about 2.5 oz.

3. The vitamin shot according to claim 1, wherein the extract comprises lavender or pomegranate.

4. The vitamin shot according to claim 1, wherein the carrier is water.

5. The vitamin shot according to claim 1, further comprising a sweetener.

6. The vitamin shot according to claim 5, wherein the sweetener is monk fruit.

7. The vitamin shot according to claim 1, further comprising caffeine.

8. The vitamin shot according to claim 7, wherein the caffeine is caffeine extracted from green tea.

9. The vitamin shot according to claim 1, further comprising at least one additive selected from the group of binders, emulsifiers, maskers, stabilizers, and preservatives.

10. The vitamin shot according to claim 1 comprising:
700-900 mcg Vitamin A;
180-450 mg Vitamin C;
20-50 mcg Vitamin D3;
15-22.5 mg Vitamin E;
90-180 mcg Vitamin K2;
1.2-3 mg Vitamin B1;
1.3-3.25 mg Vitamin B2;
16-40 mg Vitamin B3:
5-12.5 mg Vitamin B5;
4.25-8.5 mg Vitamin B6;
30-75 mcg Vitamin B7;
400-600 mcg Vitamin B9;
6-15 mcg Vitamin B12;
75-130 mg Calcium;
4.5-18 mg Iron;
112.5-187.5 mcg Iodine;
40-50.4 mg Magnesium;
8.25-11 mg Zinc;
27.5-65 mcg Selenium;
450-900 mcg Copper;
0.5-2.2 mg Manganese;
25-53 mcg Chromium;
35-105 mg Phosphorous;
27.5-82.5 mg Choline;
wherein the carrier comprises water;
caffeine; and
a sweetener.

11. The vitamin shot according to claim 1 comprising:
900 mcg Vitamin A;
180 mg Vitamin C;
25 mcg Vitamin D3;
15 mg Vitamin E;
120 mcg Vitamin K2;
1.2 mg Vitamin B1;
1.3 mg Vitamin B2;
16 mg Vitamin B3:
5 mg Vitamin B5;
4.25 mg Vitamin B6;
30 mcg Vitamin B7;
400 mcg Vitamin B9;
6 mcg Vitamin B12;
130 mg Calcium;
4.5 mg Iron;
37.5 mcg Iodine;
40 mg Magnesium;
8.25 mg Zinc;
27.5 mcg Selenium;
450 mcg Copper;
0.58 mg Manganese;
26.25 mcg Chromium;
40 mg Phosphorous;
55 mg Choline;
wherein the carrier comprises water;
wherein the caffeine is an extract of green tea; and
a sweetener.

12. The vitamin shot according to claim 1,
900 mcg Vitamin A;
180 mg Vitamin C;
25 mcg Vitamin D3;
15 mg Vitamin E;
120 mcg Vitamin K2;
1.2 mg Vitamin B1;
1.3 mg Vitamin B2;
16 mg Vitamin B3:
5 mg Vitamin B5;
4.25 mg Vitamin B6;
30 mcg Vitamin B7;
400 mcg Vitamin B9;
6 mcg Vitamin B12;
130 mg Calcium;
4.5 mg Iron;
37.5 mcg Iodine;
40 mg Magnesium;
8.25 mg Zinc;
27.5 mcg Selenium;
450 mcg Copper,
0.58 mg Manganese;
26.25 mcg Chromium;
40 mg Phosphorous;
55 mg Choline;
Lavender extract;
Pomegranate extract;
Monk fruit;
Citric acid
wherein the carrier comprises water;
caffeine; and
a sweetener.
